(19) United States
(12) Patent Application Publication
(10) Pub. No.: US 2010/0202920 A1
(43) Pub. Date: Aug. 12, 2010

(54) MACHINE AND METHOD FOR WASHING AND/OR DISINFECTING MEDICAL INSTRUMENTS

(75) Inventors: Andreas OTTE, Wiesbaden (DE); Karl-Guido MERK, Wedel (DE); Heinz JACQUI, Bad Schwartau (DE)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS WINTER & IBE GMBH, Hamburg (DE)

(21) Appl. No.: 12/723,934

(22) Filed: Mar. 15, 2010

Related U.S. Application Data
(63) Continuation of application No. PCT/EP2008/007711, filed on Sep. 16, 2008.

(30) Foreign Application Priority Data
Sep. 19, 2007 (DE) 10 2007 044 957.9

(51) Int. Cl. A61L 2/28; A61L 2/18

(52) U.S. Cl. 422/28; 422/119

(57) ABSTRACT

A method for washing and/or disinfecting medical instruments is provided. The method including: extracting a sample of an agent used in the washing, disinfecting and/or rinsing of the medical instruments in a machine used for the washing, disinfecting and/or rinsing and prior to the washing, disinfecting and/or rinsing with the agent; and subjecting the sample to chemical and/or biological testing in the machine used for the washing, disinfecting and/or rinsing and prior to the washing, disinfecting and/or rinsing with the agent.

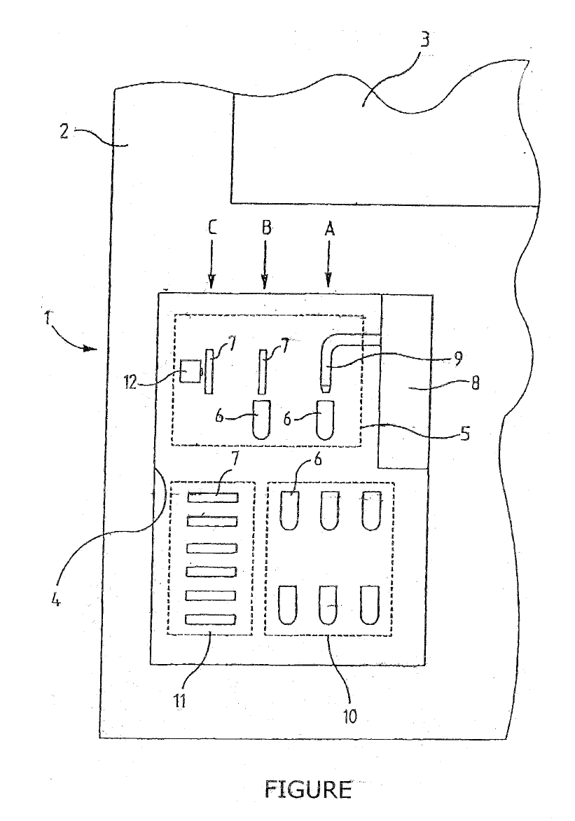

### BACKGROUND

1. Field of the Invention

The present invention relates generally to a machine for washing and/or disinfecting medical instruments and more particularly to a machine for washing and/or disinfecting medical instruments using rinsing water.

2. Prior Art

Machines of this type are used in particular for cleaning endoscopes. Washing is normally carried out first, with subsequent disinfecting. It is always necessary to rinse at the end of the cleaning procedure with clear water in order to, for example, remove cleaning or disinfection agent residues that could interfere with the later use of the instrument in a patient.

Disinfection agents and cleaning agents can be faulty, for example due to concentration variations or contamination. Rinsing water, namely clear water, is susceptible to germs. It can be insufficiently sterilized or can be germ-infested following sterilization, for example when standing in contact with air or through contact with germ-infested objects.

Such faults in the agents used for washing, disinfecting or rinsing will result in an incorrect cleaning and/or disinfecting, and therefore in substantial safety risks for the patients to be treated.

### SUMMARY

It is therefore the purpose of the present invention to increase the safety of a machine for washing and/or disinfecting medical instruments.

According to a method for washing and/or disinfecting medical instruments, the agents used for washing, disinfecting or rinsing are tested in the machine. Germ-infestation problems are solved in this way as it is always known whether one is working with faultless agents. Washing and cleaning agents can for example be checked for concentration, pH etc.

If testing took place externally, e.g. outside of the machine this could lead to incorrect test results in that the sample could become contaminated upon extraction from the machine. According to the method for washing and/or disinfecting medical instruments, testing is therefore carried out with a testing means that is located inside the closed and sealed machine, which carries out the test process independently, namely without interference from the outside. In this way it is ensured that any adulteration detected during the test originates from the machine, and not from the outside or from a machine operator. False positive results are therefore excluded.

A test is preferably carried out each time that instruments have been treated inside the machine, so that correct cleanliness and sterility is guaranteed for every instrument treated, or so that it can be ensured that unclean or germ-infested instruments are not used if faults are detected.

The sterility of the rinsing water can be tested, a parameter that is of crucial importance, as non-sterile rinsing water would destroy the result of all prior efforts.

A rapid test can be used, with which the usual test period of, for example, 24 hours that would be required by an external test laboratory is reduced to, for example, 15 minutes, which can for example be utilized for dripping off or drying inside the machine.

### BRIEF DESCRIPTION OF THE DRAWINGS

These and other features, aspects, and advantages of the apparatus and methods of the present invention will become better understood with regard to the following description, appended claims, and accompanying drawings where:

The FIGURE shows a partial front view of a machine for washing and/or disinfecting medical instruments.

### DETAILED DESCRIPTION OF THE PREFERRED EMBODIMENT

The FIGURE shows a partial front view of a washing machine 1 with a front surface of its housing 2, in which a flap 3 is located, enabling access to a loading chamber similar to that of a domestic dishwasher. Inside the same, all kinds of medical instruments, such as for example, endoscopes can be placed, for example, in suitable wire baskets.

The washing machine 1 serves, for example, for cleaning and disinfecting in the usual way. For this the washing machine can be designed substantially like a normal domestic dishwasher, namely with a fresh water inlet and a waste water outlet as well as with a circulation pump for operating pressure nozzles, with which the instruments are sprayed. Internal channels of the instruments can also be connected to pressure lines if necessary.

Cleaning agents to be added to the water for cleaning can be stored in the tanks located inside the housing. Following a thorough cleaning, for example using several steps, disinfection is carried out with the aid of a disinfection agent that is also added to the water from storage tanks

The washing machine can also be designed only for cleaning or only for disinfecting. Irrespective of whether only cleaning or only disinfecting is carried out, or whether both steps are carried out one after the other, clear water must finally always be used for rinsing. This rinsing water normally comes from the fresh water connection. When the fresh water is germ-infested, the medical instruments can become infested with germs, even after they have previously been correctly sterilized. The sterility of the rinsing water must therefore be ensured.

If one works with clean fresh water from a municipal water supply high sterility can normally be guaranteed, although it is not always present. Bio-films present in in-house installations can also result in contamination. Furthermore even extremely sterile fresh water can become germ-infested inside the machine, for example, when water stands inside the same at higher temperatures over a period of time or comes into contact with germs in some other way.

In order to ensure that a possible germ infestation of the instruments to be treated inside the machine 1 is detected, the rinsing water is checked for sterility.

For this, a test means 5 is located inside the machine interior behind an opening 4 in the housing 2, which is closed and sealed, preferably with a cover (not shown).

The test means 5 is illustrated schematically in the FIGURE and surrounded by a broken line.

In the embodiment shown, a normal commercial rapid germ test is used involving test tubes 6 containing a reagent, for example in a dried form. Furthermore a test strip 7 is used, which is submersed following the reaction of the water with the reagent and then optically read and, for example, checked for color changes.

The test means 5 shown, runs automatically, for example by means of a controller via a data processing unit (not shown), and for example, comprises controlled manipulators (not shown) for handling the test tubes 6 and the test strips 7. The test means 5 can always be started when a cleaning and/or disinfecting process has been completed with a rinsing process.

The rinsing water dripping off the instruments is extracted from a collection trough 8 that is similar to that of a domestic dishwasher by means of a sampling line 9 and added to a test tube 6 located in position A, which has previously been taken from a reagent supply 10 that can be equipped with six test tubes in the embodiment shown in the FIGURE. Substantially larger quantities are also possible.

Once the test tube 6 has been filled from the sample line 9 the test tube 6 is moved to the left by one position to position B below a test strip 7 held there as shown in the FIGURE, the same having been taken from test strip supply 11 in which a corresponding number of test strips can be stored. The test strip 7 is now submersed in the filled test tube 6 and is then moved one step to the left into position C in front of a detector 12, which can, for example, recognize a color change on the test strip in a photometric way.

The detector 12 supplies its measurement result to a data processing unit in a way not shown, which is installed in the washing machine 1, or to the outside via data lines, for example to a main hospital computer. The measurement result from the detector 12 is examined and evaluated in order to find out whether the tested water is germ-infested or not. The evaluating data processing unit can issue a notification when a germ infestation is detected, for example by means of an acoustic or optical warning signal or the like.

The opening 4 is opened only to top up the reagent supply 10 and test strip supply 11 and to remove the used test tubes 6 and test strips 7 that can, for example, be deposited in the supply areas 10 and 11 after use. The opening 4 is then closed and sealed again, so that, for example, six tests can be carried out in the embodiment shown without the same being corrupted by germs entering from the outside. Greater quantities of test material would of course enable substantially more tests without maintenance.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A machine for washing and/or disinfecting medical instruments, the machine comprising:
a housing; and
a test means disposed in the housing in a closed and sealed manner for chemical and/or biological testing of a sample of an agent used in the washing, disinfecting and/or rinsing of the medical instruments.

2. The machine of claim 1, wherein the test means further comprises a means for extraction of the sample of the agent used in the washing, disinfecting and/or rinsing of the medical instruments.

3. The machine of claim 2, wherein the means for extraction comprises a sample line.

4. The machine of claim 1, wherein the test means comprises one or more test strips.

5. The machine of claim 2, wherein the test means comprises an optical detector for reading results of the one or more test strips.

6. The machine of claim 1, wherein the sample is water and the test means is configured to test the sterility of the water.

7. The machine of claim 1, where the test means is configured for a rapid testing of the sample.

8. A method for washing and/or disinfecting medical instruments, the method comprising:
extracting a sample of an agent used in the washing, disinfecting and/or rinsing of the medical instruments in a machine used for the washing, disinfecting and/or rinsing and prior to the washing, disinfecting and/or rinsing with the agent; and
subjecting the sample to chemical and/or biological testing in the machine used for the washing, disinfecting and/or rinsing and prior to the washing, disinfecting and/or rinsing with the agent.

9. The method of claim 8, further comprising issuing a warning to a user of the machine if results of the testing are negative.

10. The method of claim 8, wherein the extracting comprises utilizing one or more test tubes operatively connected to a sample line for filling the one or more test tubes with the sample.

11. The method of claim 10, wherein the testing comprises exposing the sample to one or more test strips and optically reading results of the one or more test strips.

12. The method of claim 11, wherein the filling of the one or more test tubes with the sample occurs at a first station, the exposing of the sample to the one or more test strips occurs at a second station and the optically reading of the results of the one or more test strips occurs at a third station.

13. The method of claim 8, wherein the testing tests for a sterility of the sample.
